(19) United States
(12) Patent Application Publication
(10) Pub. No.: US 2024/0079099 A1
(43) Pub. Date: Mar. 7, 2024

(54) INFERRING DEVICE, TRAINING DEVICE, INFERRING METHOD, METHOD OF GENERATING REINFORCEMENT LEARNING MODEL AND METHOD OF GENERATING MOLECULAR STRUCTURE

(71) Applicant: Preferred Networks, Inc., Tokyo-to (JP)

(72) Inventors: Ryuichiro ISHITANI, Tokyo-to (JP); Toshiki KATAOKA, Tokyo-to (JP)

(73) Assignee: Preferred Networks, Inc., Tokyo-to (JP)

(21) Appl. No.: 18/506,509

(22) Filed: Nov. 10, 2023

Related U.S. Application Data
(63) Continuation of application No. PCT/JP22/09553, filed on Mar. 4, 2022.

(30) Foreign Application Priority Data
May 26, 2021 (JP) 2021-088160

(51) Int. Cl. G16C 20/70; G16C 20/50

(52) U.S. Cl. CPC G16C 20/70; G16C 20/50

(57) ABSTRACT

An inferring device comprises one or more memories and one or more processors. The one or more processors execute decision of an action based on a tree representation including a node and an edge of a molecular graph, and a trained model trained through reinforcement learning, and execute generation of a state including information on the molecular graph based on the action, wherein the edge has connection information on the nodes.

### CROSS REFERENCE TO THE RELATED APPLICATIONS

This application is continuation application of International Application No. JP2022/009553, filed on Mar. 4, 2022, which claims priority to Japanese Patent Application No. 2021-088160, filed on May 26, 2021, the entire contents of which are incorporated herein by reference.

### FIELD

The present disclosure relates to an inferring device, a training device, an inferring method, a method of generating a reinforcement learning model, and a method of generating a molecular structure.

### BACKGROUND

In these days, in a field related to development of new drugs, and materials, methods of inferring candidates for a compound by using a trained model are studied actively. In these studies, methods are adopted in which a chemical formula of a compound is represented by a character string or a graph through a method of converting the chemical formula into the character string or a method of converting the chemical formula into the graph, and the representation of the character string or the graph is subjected to reinforcement learning.

However, in the method of converting the chemical formula into the character string, invalid molecules are often generated, and on the other hand, in the method using the graph, invalid molecules are not generated but it is difficult to infer molecular structures according to purposes.

### BRIEF DESCRIPTION OF DRAWINGS

FIG. 1 is a diagram schematically illustrating a concept of tree decomposition of a graph according to one embodiment;

FIG. 2 is a diagram schematically illustrating one example of a tree with site information according to one embodiment;

FIG. 3 is a diagram schematically illustrating one example of a tree with site information according to one embodiment;

FIG. 4 is a diagram schematically illustrating one example of a tree with site information according to one embodiment;

FIG. 5 is a diagram schematically illustrating one example of a tree with site information according to one embodiment;

FIG. 6 is a block diagram illustrating one example of an inferring device according to one embodiment;

FIG. 7 is a block diagram illustrating one example of an inferring unit according to one embodiment;

FIG. 8 is a flow chart illustrating processing of an inferring device according to one embodiment;

FIG. 9 is a diagram schematically illustrating reinforcement learning according to one embodiment;

FIG. 10 is a diagram schematically illustrating an environment and a state according to one embodiment;

FIG. 11 is a diagram schematically illustrating an action according to one embodiment;

FIG. 12 is a block diagram illustrating one example of a training device according to one embodiment;

FIG. 13 is a block diagram illustrating one example of a training unit according to one embodiment;

FIG. 14 is a diagram illustrating one example of formation of models according to one embodiment;

FIG. 15 is a flow chart illustrating processing of a training device according to one embodiment; and

FIG. 16 is an example of a hardware implement of one embodiment.

### DETAILED DESCRIPTION

According to one embodiment, an inferring device comprises one or more memories and one or more processors. The one or more processors execute decision of an action based on a tree representation including a node and an edge of the molecular graph, and a trained model trained through reinforcement learning, and execute generation of a state including information on a molecular graph based on the action, wherein the edge has connection information on the nodes.

Hereinafter, embodiments of the present invention will be explained with reference to the drawings. The explanations of the drawings and the embodiments are presented by way of example only, and are not intended to limit the present invention.

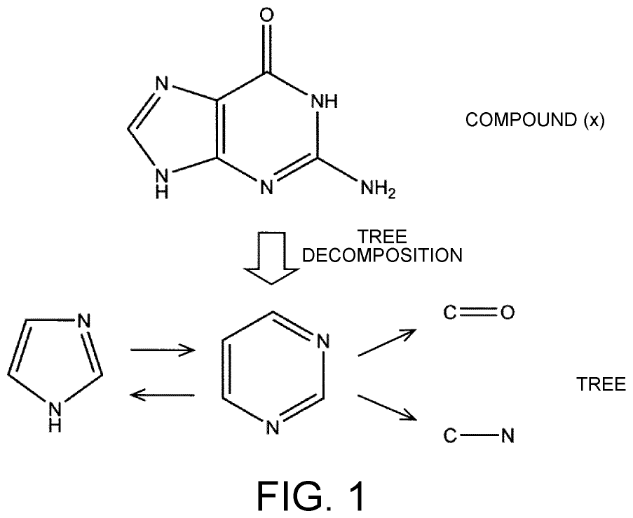

FIG. 1 is a conceptual diagram regarding tree decomposition of a graph according to one embodiment in the present disclosure. In the present embodiment, a compound x represented by a graph is subjected to tree decomposition as illustrated in the diagram. Here, the tree decomposition means a method of mapping a graph representation of a molecular structure (molecular graph) into a tree representation. This tree decomposition does not convert a graph into a tree representation per atom, but it converts a graph into a tree representation per molecule, as an example.

For example, the compound as illustrated in an upper diagram is converted as illustrated in a lower diagram, to thereby acquire a tree structure. In particular, the compound illustrated in the upper diagram has a ring structure (loop structure), so that a method of tree decomposition in which this ring structure is converted as one node, is used. On the other hand, when the conversion as above is performed, it is not possible to uniquely perform reverse conversion from the tree representation into the original graph representation of the compound x. Accordingly, in the present embodiment, a concept of tree decomposition with site information is used.

The tree decomposition with site information is a representation including information indicating what kind of connection state is established between nodes in the tree representation, with respect to the tree representation illustrated in the lower part of FIG. 1 that is obtained by converting the graph while setting the ring structure to the molecule.

In the explanation below, words of node and edge are used. In the present disclosure, they mainly indicate a node and an edge in the tree representation, but in the description regarding before and after the tree decomposition, the node and the edge in the graph representation and the node and the edge in the tree representation will be replaced as appropriate.

In a junction tree representation, each node is a node of any one of a singleton node, a bond node, and a ring node.

The singleton node indicates a node corresponding to an atom being a branch point in the case of decomposing the graph of a compound molecule into a tree. The branch point in the graph representation becomes the singleton node when the branch point does not belong to a ring structure.

The bond node expresses two atoms which are covalently bonded as one node. However, a covalent structure included in the ring node to be described below is described as (a part of) the ring node, and is not converted into the bond node.

The ring node is a node corresponding to the ring structure in the case of decomposing the graph representation of a compound molecule into a tree. Representative examples of the compound expressed as the ring include benzene, pyridine, and pyrimidine, or cyclobutadiene, cyclopentadiene, pyrrole, cyclooctatetraene, and cyclooctane, but the compound is not limited to them and only needs to be a cyclic one.

Further, as the connection between the nodes described above, there are four kinds of bond-bond, bond-singleton (or singleton-bond), bond-ring (or ring-bond), and ring-ring.

The bond-bond is a case where two bonds are connected to each other.

The bond-singleton is a case where one bond is connected to a singleton being a branch point.

The ring-bond is a case where one bond is connected to a ring.

The ring-ring is a case where two rings are directly connected. In this case, there are a condensing (connecting by sharing one bond) case and a spiro bonding (connecting by sharing only one atom) case.

When general tree decomposition is performed on the graph representation of a compound, information on a connection position or the like is lost. Therefore, restoration with uniqueness from the tree representation to the graph information is impossible with only the tree information. In the present embodiment, to uniquely convert between the tree representation and the graph representation in a bi-directional manner, node connection information indicating the relationship between the nodes connected to each other is given, as information about a site, to the tree representation. The information about the site will be described as site information hereinbelow. This connection information includes, as an example, at least either of information on the position where the nodes are connected and information on a direction in which the nodes are connected. The connection information is only required to include information used for the restoration from the tree representation to the graph information.

The site information is information indicating a relationship of how the nodes in the tree representation are connected in the original molecular structure. The general tree decomposition is irreversible, but the use of this site information enables to ensure reversibility of performing reverse conversion from the tree representation into the graph representation. Further, the tree representation added with the site information will be described as a tree representation with site information.

(Tree Representation with Site Information)

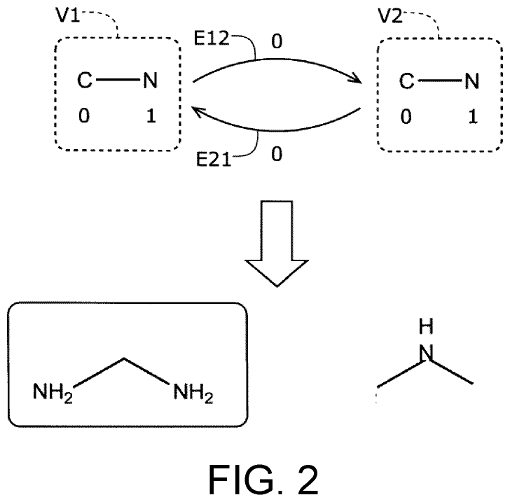

FIG. 2 is a diagram schematically illustrating one example of a tree with site information. FIG. 2 illustrates the site information regarding the bond-bond connection, but the bond-singleton case can be similarly processed. In this diagram, two nodes (nodes V1, V2) of C-N are connected by directed edges added with site information, for example. The directed edges are indicated by arrow marks in the diagram.

In the node V1, 0 is assigned as an identifier to a carbon atom (C), and 1 is assigned as an identifier to a nitrogen atom (N). In the node V2, 0 is assigned to C and 1 is assigned to N, in a similar manner. The method of adding the identifiers is described as an example, and any method can be employed as long as it uniquely adds an identifier to an atom or a molecule in an appropriate manner within a node. For example, the identifier may be added in an alphabetical order, in an order of atomic number, in an order of molecular weight, in a random order within a node, or the like, or it may also be added by another method. Note that it is possible to design such that as the identifier added in a certain node, the same one is added until when training or inference is terminated. Accordingly, it is preferable to employ a method having some kind of regularity in which an identifier is uniquely added to a certain node, but the method is not limited to this.

Edges E12, E21 connect between the node V1 and the node V2. The edge E12 is an edge that connects the node V1 to the node V2, and the edge E21 is an edge that connects the node V2 to the node V1. To the edge E12, 0 is added as the site information. This site information means that “0” in the node V1 is connected to the node V2. In like manner, 0 is given as the site information to the edge E21, and this site information means that “0” in the node V2 is connected to the node V1.

In the connection of mutual bonds as described above, there can be considered two patterns of molecules of CH₃NHCH₃ and NH₂CH₂NH₂, as illustrated in a lower diagram, but by defining the site information as described above, it is possible to uniquely perform reconfiguration to obtain NH₂CH₂NH₂, as illustrated in a lower left diagram, with the use of the tree with site information.

Note that in the above description, the site information is set to indicate the identifier of the connection-destination node, but it may indicate, on the contrary, a connection portion in the connection-source node. Specifically, it is possible to define that the node V1 is connected to “0” in the node V2.

Further, as another example, it is also possible to describe identifiers to be connected in both nodes. For example, when, in a case where there are a node A and a node B, an identifier “0” of the node A and an identifier “1” of the node B are connected, site information indicating “0, 1” may be added to an edge from the node A to the node B, and site information indicating “1, 0” may be added to an edge from the node B to the node A.

Further, although the graph-graph case is indicated in the above description, the singleton case can be similarly defined. In this case, the site information is not required but may be added in implementation.

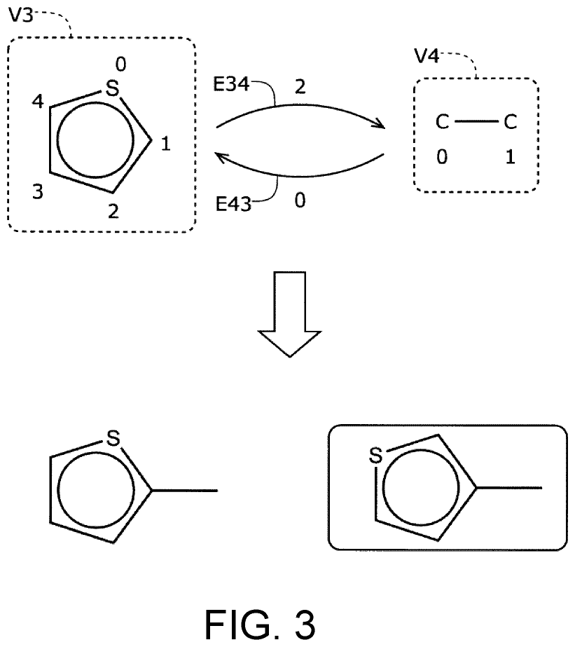

FIG. 3 is a diagram schematically illustrating another example of the tree with site information. FIG. 3 illustrates the site information regarding the ring-bond connection. A node V3 is a ring node, and a node V4 is a bond node.

To identifiers of the node V3, numbers are given in such a manner to pass through all of the atoms starting from a predetermined atom in the ring of the graph of the node V3. As illustrated in FIG. 3, identifiers 0, 1, . . . , 4 are given clockwise in order starting from S in the diagram, for example. For the node V4, identifiers are given similarly to the case in FIG. 2. Similarly to the above-described case, the method of giving the identifiers is described as an example, and not limited to this method.

To an edge E34 from the node V3 toward the node V4, “2” being the number of the atom to be connected in the node V3 is added, and to an edge E43 from the node V4 toward the node V3, “0” being the number of the atom to be connected in the node V4 is added.

In the case of having the above site information, it is possible to uniquely restore a graph in a lower right diagram, among a plurality of connection states of the nodes V3, V4, from the tree representation with site information. In the case of connection of the ring node and the bond node, by adding the position of the atom to be connected in each of the nodes as the site information as above, it becomes possible to uniquely restore the atomic graph (graph representation) from the tree information (tree representation).

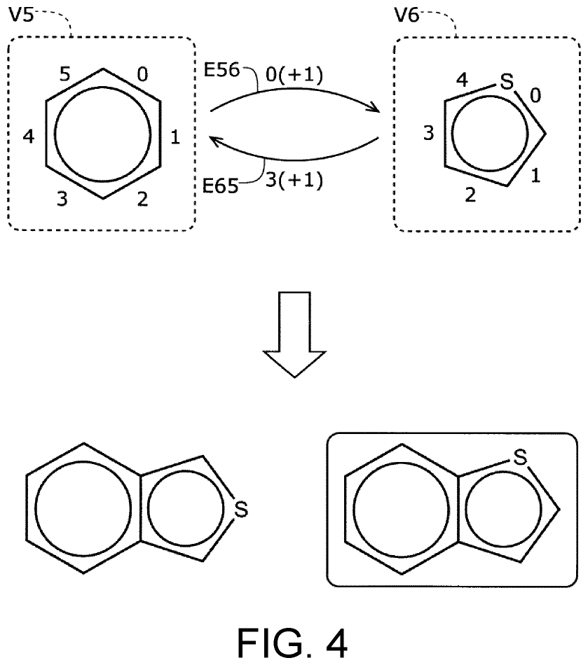

FIG. 4 is a diagram schematically illustrating still another example of the site information. FIG. 4 illustrates the site information regarding the condensation connection, in the connections of the ring node and the ring node. For example, a node V5 is a 6-membered ring aromatic compound, and a node V6 is a 5-membered ring aromatic compound.

In the node V5, identifiers 0, 1, . . . , 5 are added clockwise in order from a certain side in the atomic graph. In the node V6, identifiers 0, 1, . . . , 4 are added clockwise in order from a certain side in the atomic graph. Unlike FIG. 3, the identifier as the site information is added not to the node of the atomic graph but to the edge of the atomic graph. Similarly to the above, the way of giving these identifiers is described as an example, and not limited to this giving method.

To an edge E56 from the node V5 to the node V6, “0(+1)” is added as the site information, and to an edge E65 from the node V6 to the node V5, “3(+1)” is added as the site information. This site information includes a position identifier (“0”, “3”, or the like) indicating the connection position of the graph in the node, and a direction identifier (“+1” or the like, which may be simply a reference numeral, and hereinafter, the direction identifier may be described also as site direction information) indicating the connection direction. S in the node V6 is a node of the atomic graph for explanation, which is added for making it easy to understand the connection state of the mutual atomic graphs. Identifiers are given to edges of the atomic graph in order from the node S of the atomic graph.

For example, the edge E56 means that the edge 0 of the atomic graph in the node V5 and the node V6 are connected based on the position identifier (0), and they are connected in a direction same as a direction in which the numbers of the identifiers of the atomic graph are given, based on the direction identifier (+1). Specifically, the node V5 at the identifier “0” is connected to the node V6 in a state illustrated in the diagram (in a not-reversed state). In like manner, based on the edge 65, the node V6 at the identifier “3” is connected to the node V5 in a state illustrated in the diagram. Accordingly, it is possible to uniquely restore the connection state as illustrated in the lower right diagram, among a plurality of connection states, from the tree representation with site information.

As another example, when the edge E65 has site information of “3(−1)”, for example, the node V6 is connected at the edge 3 of the atomic graph indicated by the position identifier (3) to the node V5 in a reverse order being a direction indicated by the direction identifier (−1). The reverse direction means that the atomic graph of the node V6 is reversed to be connected in FIG. 4, for example.

In the case of condensation connection of mutual ring nodes, by providing the position identifier indicating the connection position and the direction identifier indicating the connection direction, as the site information added to the directed edges, as described above, the graph can be restored more appropriately.

Note that regarding the ring node, different descriptions may express the similar meaning, due to symmetry of a molecular structure. For example, in the structure in FIG. 4, the case where “3(+1)” is added to the edge E65 and the case where “1(−1)” is added to the edge E65 may indicate the same structure. In such a case, either of the expressions may be used as long as it is possible to uniquely restore the graph information from the tree representation with site information.

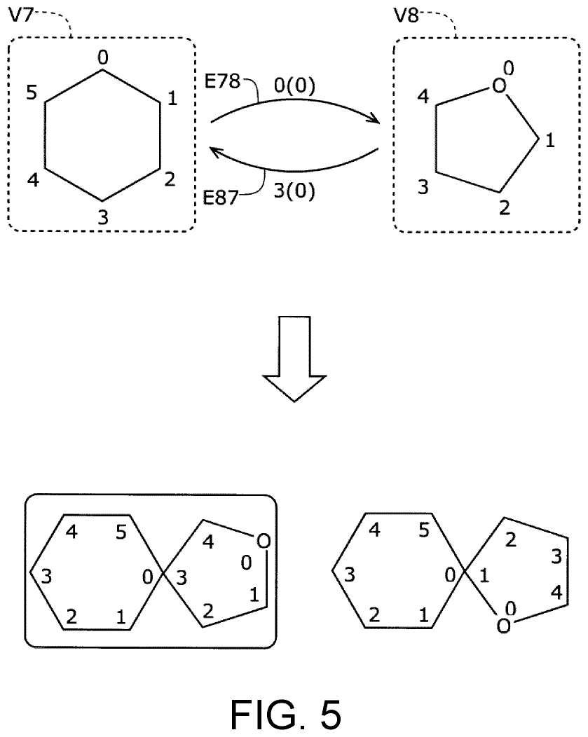

FIG. 5 is a diagram schematically illustrating still another example of the site information. FIG. 5 illustrates the site information regarding the spiro connection, in the connections of the ring node and the ring node. For example, a node V7 is a 6-membered ring aromatic compound, and a node V7 is a 5-membered ring aromatic compound.

In the case of the spiro connection, the site direction information is defined as “0”, for example. When the restoration from the tree representation with site information indicating the ring node and the ring node to the graph is performed, the direction identifier (the site direction information) may be referred to at first. When the direction identifier is 0, designated atoms are connected to each other to restore the graph information, as illustrated in FIG. 5. On the other hand, when the direction identifier is ±1, edges are connected to each other based on the case in FIG. 4, to thereby restore the graph information.

For example, in FIG. 5, site information on an edge E78 includes the position identifier of 0, site information on an edge 87 includes the position identifier of 3, and direction identifiers of both the edges are 0. Accordingly, the connection of these is determined as the Spiro connection, and it is possible to restore the atomic graph as illustrated in a lower left diagram, in which the node of “0” of the molecular graph of the node V7 and the node of “3” of the molecular graph of the node V8 are connected.

As described above, the description of identifier is cited as an example, and not limited to this. Further, the direction identifier may be conceptually given to not only the ring node but also the bond node and the singleton node in a similar manner. In this case, the direction identifier of the bond node and the singleton node may be constantly fixed to the same value, for example, “0”, or it may be ignored (not considered) in training, inference, and so on.

In the present disclosure, the graph information indicating the molecular structure of the compound is converted into the tree representation with site information, training of a model regarding the molecular structure is executed through reinforcement learning based on the information, and the trained model after the training is used to infer the graph information indicating the molecular structure of the compound. The tree decomposition with site information is executed through the conversion into the tree representation in which the group in the molecular structure of the compound is set to the node, as explained above. Hereinafter, reverse processing of the conversion into the tree representation, will be described as assemble. The assemble is processing of uniquely generating the graph of the compound based on at least one of the above-described respective methods, from the tree representation with site information in which the group in the molecular structure of the compound is set to the node.

(Inferring Device)

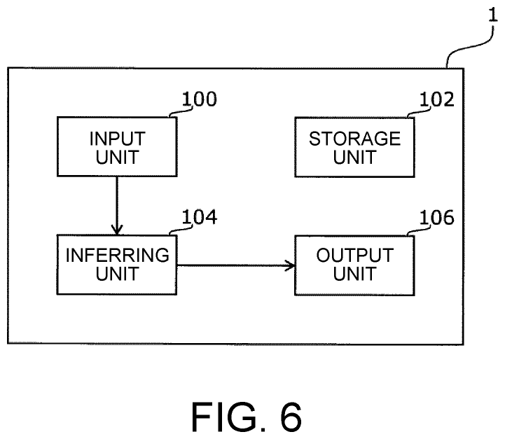

FIG. 6 is a block diagram illustrating one example of an inferring device according to one embodiment. An inferring device 1 includes an input unit 100, a storage unit 102, an inferring unit 104, and an output unit 106. The inferring device 1 uses a trained model trained through reinforcement learning to output an appropriate compound. Note that the inferring device 1 may further include another element that is not illustrated, in order to realize an operation.

The input unit 100 includes an input interface, for example, and accepts an input with respect to the inferring device 1. The input information is, for example, information on a molecule to be a starting point, or the like. The inferring device 1 generates, based on the information on the molecule to be the starting point or the like, information on a compound with maximized reward through reinforcement learning using a neural network model such as a deep neural network. This trained model may be learned in advance by using at least either supervised learning using known molecular structures or unsupervised learning. The information on the molecule to be the starting point or the like may be set to fixed molecular information such as, for example, CH₃CH₃ (ethane) molecule or benzene, and as another example, it may also be information on a molecule or the like that is desirable to be included in a part of an inferred molecular structure. Further, it is also possible that a molecule configuring any node registered in a dictionary as a node of the tree representation with site information is randomly extracted, and the extracted molecule is set to the starting node.

The storage unit 102 stores information that is required for the operation of the inferring device 1. The storage unit 102 stores at least one of, for example, information such as hyperparameters and parameters required for the inference, information obtained in the interim process of the inference, and information such as a program required for the operation of the inferring device 1. Accordingly, although not illustrated, the storage unit 102 is appropriately connected to each block that transmits/receives data.

The inferring unit 104 appropriately converts the compound information accepted by the input unit 100 into a tree representation with site information, and infers a molecular structure by using a trained model trained through reinforcement learning, while using the information as information regarding an environment and a state. The molecular structure is acquired by the above-described tree representation with site information, for example. In addition, the inferring unit 104 may assemble and output the inferred tree representation with site information.

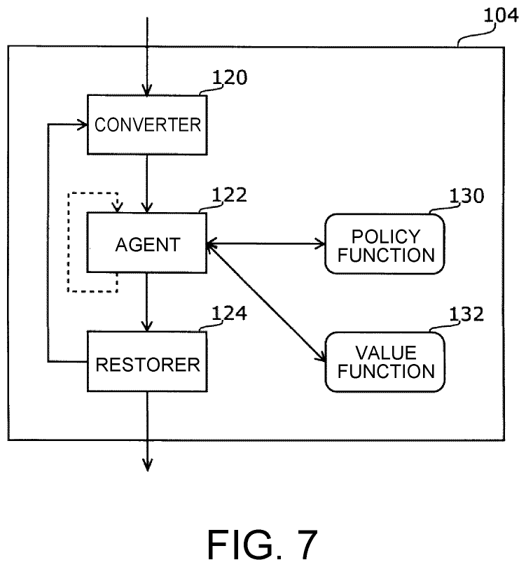

FIG. 7 is a block diagram illustrating one example of the inferring unit 104. The inferring unit 104 includes, for example, a converter 120, an agent 122, and a restorer 124.

The converter 120 performs tree decomposition on information or the like regarding a graph of the molecule or the like input in the inferring unit 104, to thereby acquire the tree representation with site information.

The agent 122 executes an action based on a policy function 130 and a value function 132 (state value) trained through reinforcement learning, to thereby generate a new state from a current state. These states are described as the tree representation with site information, as described above.

The agent 122 may acquire the policy and the value by using the trained model based on the tree representation with site information, for example. The agent 122 may also determine whether or not the new tree representation with site information acquired by the policy is appropriate as a molecular structure. In this case, it is possible to configure that the value is varied depending on whether or not the tree representation with site information has an appropriate molecular structure, in the value function 132. The agent 122 may perform the reinforcement learning so as to increase a reward or a value, when the tree representation with site information in a state of being updated by the policy is appropriate as the molecular structure.

The agent 122 may convert the tree representation with site information into a hidden vector representation according to need. The policy and the value may be acquired by using the trained model based on the converted hidden vector representation. In this case, it is possible to provide an encoder that converts the tree representation with site information into the hidden vector representation, according to need. The encoder and the models that acquire the policy and the value are neural network models, for example, and these neural network models may be trained by a training device to be described later.

The agent 122 may further determine whether the tree representation with site information is restored as an appropriate molecular structure (graph representation). When the restoration cannot be realized, the update may be rejected to select a different state transition as an action.

Note that the agent 122 may also be configured as a model that infers the tree representation with site information based on the state transition as described above, and further includes a unit similar to a training unit in the training device to be described later, so that the reinforcement learning is performed in addition to the performance of inference. For example, the inferring device 1 may include Actor and Critic in the training device, to thereby execute the inference of molecular graph while performing training of parameters and the like regarding the model. As described above, it is possible to perform the inference while performing the reinforcement learning.

The restorer 124 assembles the tree representation with site information acquired by the agent, to restore the molecular graph. The molecular graph restored by the restorer 124 is re-input in the converter 120 until when a termination condition is satisfied, and the inference is executed repeatedly.

If the reward can be obtained appropriately, the agent 122 may recursively infer the tree representation with site information until when the termination condition is satisfied, as indicated by a dotted line. For example, when the reward can be acquired from the tree representation with site information, the inference may be repeatedly executed by the agent 122 without performing the assemble.

The recursive processing may have the following configuration, for example. The agent infers a probability of taking each action based on the policy, and decides the action through sampling. Subsequently, the agent 122 changes a state based on the decided action. The agent 122 calculates a reward of the changed state. These pieces of processing are executed a plurality of times. In a process of executing the pieces of processing a plurality of times, neural network models regarding the policy and the value function are trained by using a pair of action and reward as training data. The inferring device 1 may repeat the inference in a manner as described above.

The restorer 124 executes the processing of assemble based on the tree representation with site information in the updated state, to thereby acquire information on a chemical formula representing a compound, a structural formula, or the like. As described above, by using the tree representation with site information, the restorer 124 can uniquely restore the graph representation representing the compound with respect to the output of the inferring unit 104.

The output unit 106 outputs the graph representation restored by the restorer 124 in the inferring unit 104 to the outside or the storage unit 102.

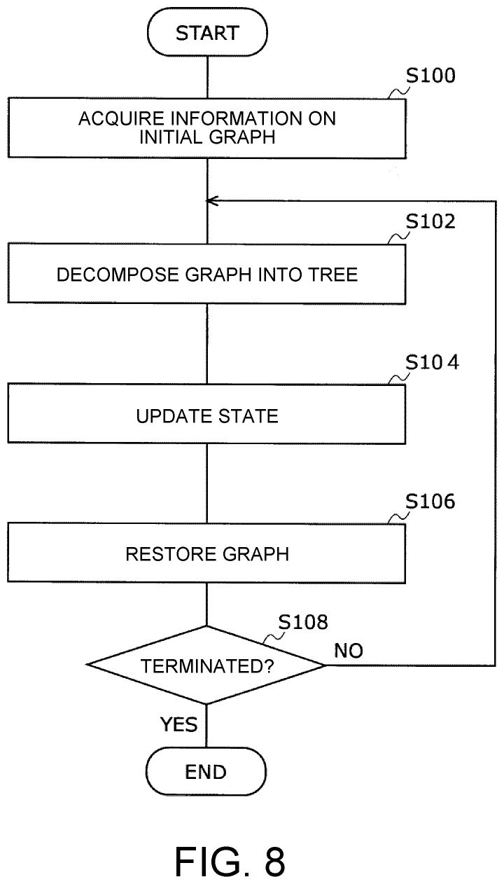

FIG. 8 is a flow chart illustrating processing of the inferring device 1 according to the present embodiment. First, a flow of the processing of the inferring device 1 will be explained by using this flow chart. Concrete implementation examples of the respective pieces of processing will be described later in detail.

First, the inferring device 1 acquires information regarding an initial graph via the input unit 100 (S100). The input information may be, for example, a node of a graph of a molecule or the like desired to be set as a starting point. Further, the input information may be information regarding a node of a graph representing a randomly-decided molecule. For example, a molecular graph desired to be included may be accepted by the input unit 100. The input information may be not information on a graph of a molecule or the like but a chemical formula or the like, and in this case, the inferring device 1 may further include a graph generating unit that converts the chemical formula or the like into graph information. These pieces of information may be appropriately stored in the storage unit 102.

Next, in the inferring unit 104, the converter 120 converts the graph information input via the input unit 100 into the tree representation with site information (S102). The converter 120 performs the conversion into the tree representation with site information that is a representation capable of being uniquely restored to the graph information, as described above. Note that it may be configured such that the tree representation with site information is acquired in the input unit 100 and the processing in the converter 120 is omitted.

Next, the agent 122 updates the tree representation with site information, to thereby infer information on a compound. The agent 122 makes a state transit based on the policy function 130 to update the state (S104). The policy function 130 is a function expressed by using a trained model trained through reinforcement learning, for example. When executing both the inference and the training through the reinforcement learning, the agent 122 determines a reward, and acquires a value based on the reward. Further, the agent 122 calculates an error of the value regarding the state transition, and updates parameters of the value and the policy. By the update based on the policy, it is inferred that what kind of molecule is connected at which position of the current molecular configuration. The method of updating the policy and the state will be explained in detail in a later-described embodiment regarding the training device.

Next, the restorer 124 assembles the tree representation with site information output by the agent 122, to thereby restore the molecular graph (S106). The restorer 124 restores the molecular graph of the compound by re-synthesizing the molecular graph based on the information on the nodes of the tree representation with site information output by the agent 122 and the site information given to the edges. Note that it may be configured such that the processing in the restorer 124 is not executed and the tree representation with site information is output as it is.

Next, the inferring unit 104 determines whether or not the update of the state, namely, the acquisition of the molecular graph or the tree representation with site information has been terminated (S108). For example, in the present embodiment, conditions such that the update of the state has been executed a predetermined number of times, a predetermined number of nodes have been added in the tree representation with site information, or the molecular graph has reached a predetermined molecular weight, may be set to the termination conditions, but the conditions are not limited to these and arbitrary termination conditions may be employed.

When the acquisition of the tree representation with site information has not been terminated (S108: NO), the inferring unit 104 repeats the processing from S102 to S106. Note that the inferring unit 104 may store the tree representation with site information before being restored in S106, in the storage unit 102, and repeat the processing of S104 by using the data. In this case, the processing of S102 can be omitted with respect to the graph representation in the updated state.

When the appropriate molecular graph has been inferred (S108: YES), the inferring device 1 outputs the molecular graph to the outside or the storage unit 102 via the output unit 106, and terminates the processing.

As described above, the inferring device 1 in the present embodiment automatically generates the molecular graph by the inferring unit 104 including the model trained through the reinforcement learning. Regarding this molecular graph, it is possible to infer a molecular graph having an appropriate structure such as a structure that satisfies conditions being purposes of generation, for example, a drug-like structure in a field of development of new drugs, or a structure suitable for an acquired material in a field of materials, by using the appropriately trained model.

(Training Device)

In order to generate such a molecular graph, there is a need to execute appropriate optimization in the reinforcement learning in the model generation. Hereinafter, an example of using PPO (Proximal Policy Optimization) as a learning method will be explained, but an algorithm to be used is not limited to this. The molecular graph may be generated by another reinforcement learning method using the tree representation with site information.

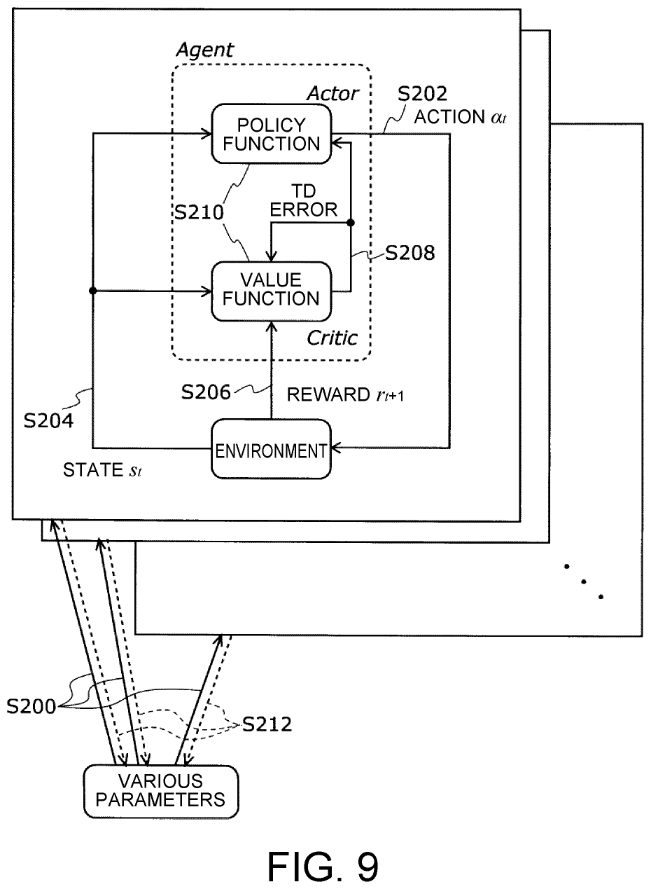

FIG. 9 is a diagram schematically illustrating an outline of an algorithm of reinforcement learning according to one embodiment. The PPO is one method of reinforcement learning using a policy gradient theorem. At a certain time t, Actor executes an action aₜ based on a policy, and a state sₜ transited by this action is output to the Actor and Critic.

In this environment, a reward rₜ₊₁ at the next time t+1 is calculated. The Critic calculates a TD error (Temporal-Difference Error) based on the reward rₜ₊₁ and a value calculated from the state sₜ by using a state value function V(s). Based on this TD error, the Actor updates a policy π_{θ}[aₜ|sₜ], and the Critic updates the state value function V(s).

For example, in the PPO, a plurality of parallel threads are first set up. The respective threads copy parameters regarding the value function and the policy function. For the calculation of the copied value function, a reward of a step of 1-unit time ahead is considered, or a reward of a step of 2-unit time or more ahead is also considered, and the copied value function and the copied policy function are updated by the aforementioned Actor and Critic.

Further, in a process that is executed synchronously or asynchronously, the value function and the policy function being the copying sources are updated based on parameter gradients in the respective updated value function and policy function, to thereby execute the update of parameters.

The above-described processing will be explained by using reference numerals in the diagram.

First, the plurality of threads copy parameters from the storage unit that stores the parameters (S200). The copied parameters are, for example, parameters regarding the policy function and the value function. Each of the policy function and the value function may be a neural network model.

Next, the Actor outputs the action aₜ based on the policy (S202). For example, the Actor selects and outputs an action capable of being inferred to have a large value, based on a probability set by the policy.

Next, this action aₜ is applied to an environment, to generate a transition state sₜ (S204). This state sₜ is output to the Actor and the Critic.

Further, in parallel with this, the reward rₜ₊₁ at the time t+1 regarding the transition state sₜ is calculated and output to the Critic (S206). At this time, it is possible to calculate not only the reward of 1-unit time ahead but also the reward of 2-unit time or more ahead.

The Critic calculates the TD error based on the input state sₜ and reward rₜ₊₁ (S208). The TD error may also be calculated based on not only the reward of 1-unit time ahead but also the reward of 2-unit time or more ahead.

The Critic updates the state value function V(s) based on the reward rₜ₊₁ and the calculated TD error, and the Actor updates the policy π_{θ}[aₜ|sₜ] based on the state sₜ and the TD error (S210). Further, as another example, the Actor may update an action value function Q(sₜ, aₜ), and it may also update the action value function Q and the policy n in a parallel manner.

This processing is repeated an arbitrary number of times, gradients are calculated based on the parameters copied in the respective threads and the updated parameters, and the gradients are reflected on the parameters being the copying sources synchronously or asynchronously (S212).

By repeatedly executing this processing, the policy and the value in the present embodiment are updated. During the processing, each of the threads appropriately stores required values such as the parameters with the use of the storage unit.

In the following, explanation will be made by citing some examples of formulae, and a formula used in the training is not limited to these. Further, although a formula is sometimes omitted, it is possible to appropriately use formulae such as, for example, generally known PPO, A3C (Asynchronous Advantage Actor-Critic), A2C (Advantage Actor-Critic), TRPO (Trust Region Policy Optimization), and DQN. Further, in order to provide randomness to the update of the policy and the like, an E-greedy method may be used.

An explanation will be made regarding how the tree representation with site information is incorporated in the network of reinforcement learning described above. More concretely, when using the PPO, the environment/state, the action, the reward, and the policy/value are defined as follows, as a not-limited example. When another method is used, it is possible to perform appropriate definition based on the method, as a matter of course.

The environment and the state may be defined by using the tree representation with site information. For example, the tree representation with site information converted into a latent representation may be set to the environment and the state, or the tree representation with site information itself as a result of decoding this latent representation may be set to the environment and the state. Further, the both pieces of information may be set to the environment and the state. Further, the molecular graph assembled from the tree representation with site information may be set to the environment and the state. In this case, it is possible to further set two pieces of information of the tree representation with site information and the molecular graph to the environment and the state, as an example.

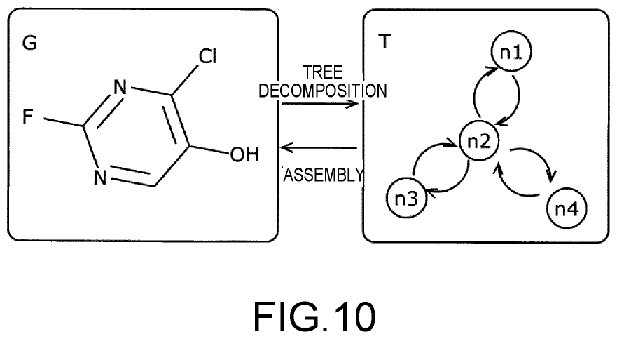

FIG. 10 is a diagram illustrating one example of the environment and the state. As illustrated in the diagram, both a tree representation with site information T and a molecular graph G restored from the tree representation with site information T, are defined as the environment and the state, as an example.

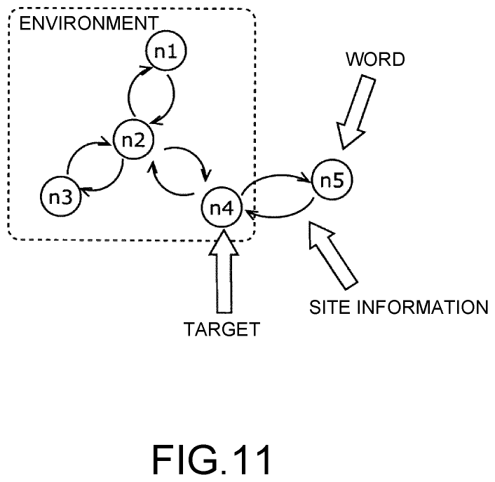

The action may be defined by using at least any one of the following four pieces of processing with respect to the environment. FIG. 11 is an outline diagram for explaining the action. These actions are executed by the Actor based on the policy.

1. Target node inference: action of selecting to which node in the current environment (tree representation with site information) the next node is connected.

As illustrated in FIG. 11, the Actor selects, for example, a node n4 as a node to which a new node is connected.

2. Word inference: action of selecting what kind of node is connected to the node selected through the target node inference.

As illustrated in FIG. 11, the word inference indicates an action of selecting information on a node n5 as a new node to be connected to the node n4, for example. The Actor selects, as the node n5, a node of one molecule or the like registered in a dictionary as node information, for example. The node registered in the dictionary is, for example, any of the singleton node, the bond node, and the ring node, and is a node indicating a molecular structure to be a candidate for each type of the nodes.

3. Site inference: action of selecting with what kind of site information the nodes selected through the target node inference and the word inference are connected to each other.

As illustrated in FIG. 11, the site information between the node n4 selected through the target node inference and the node n5 selected through the word inference is decided. The Actor decides, based on the information on the node n4 and the information on the node n5, the site information given to edges between the node n4 and the node n5.

4. Stop inference: action of stopping the addition of nodes.

The Actor executes inference regarding decision whether or not the action is performed with respect to the environment. This inference is executed based on a stop condition obtained through a probability distribution, for example. In the inferring device 1, the processing may be terminated by setting this condition to the termination condition (S108).

These actions are inferred when the Actor inputs the latent representation of the tree representation with site information indicating the environment, in the model (for example, a neural network model) describing the policy to be a target of training. When the latent representation is input in this model, the probability distributions regarding the above-described four actions are respectively output. The Actor decides the respective actions based on the probability distributions output from the model. A more concrete configuration of the model will be explained together with a later-described model regarding the value.

Based on the above-described decided action with respect to the environment, a new state is generated. In the training, a reward is calculated based on the decided action.

For example, it is assumed that the action indicating that the node n5 is connected to the node n4 based on the decided site information, is decided as illustrated in FIG. 11. In this case, with respect to the nodes n1 to n4 being the environment and the edge information with site information connecting the nodes, the node n5 is connected to the node n4 via the decided edges with site information.

There is a possibility that this tree representation cannot be appropriately restored up to the molecular graph by the constraint of valence or conformation. Accordingly, the training device may determine whether the acquired tree representation with site information can be appropriately assembled (restored to a graph representation representing a molecule). When, as a result of this determination, the assemble cannot be realized, a minus reward may be set to the action. Further, the minus reward may be set in order not to generate such a state, and at the same time, in order not to set such a state as the next environment, the Actor may generate a state again according to the policy, as described above.

The reward is set in various ways other than the above. Two examples will be cited as not-limited examples. The examples are of the case where a molecular graph regarding the development of new drugs is generated, and if a molecular graph regarding another purpose is generated, it is desirable to set a reward according to the purpose.

As a first example, a fat-soluble score (Log P) of a molecule corresponding to a generated state may be used as a reward. It is often the case where the fat-soluble score of a basic molecular structure is already calculated experimentally, and thus can be acquired relatively easily. Further, it is also possible to infer the fat-soluble score by using a trained model acquired through machine learning, for example, based on data already acquired experimentally. The fat-soluble score can be acquired by using a trained model by inputting a molecular graph in the model, for example. Further, it is also possible to generate, through training, a model of inferring the fat-soluble score from not the molecular graph but the latent representation.

As a second example, it is possible to set a degree of docking between the generated molecular graph and a substance desirable to be bonded to a molecule being an inference target, or the like, as the reward. As an example, it is possible that a docking score between predetermined protein and the generated molecular structure is calculated in the training, and this docking score is set to the reward.

The docking score may be calculated by executing a docking simulation between the predetermined protein and the generated molecular structure. In this case, the training device acquires the molecular graph from the state generated by applying the action decided by the Actor to the environment. Further, the docking simulation between this molecular graph and the predetermined protein is executed to acquire the reward. For the docking simulation, it is possible to use, for example, generally known software, program, or the like.

For example, in a molecular structure added with the node n5 as in FIG. 11, a docking simulation of rotating a bonded portion between the node n4 and the node n5 may be executed, and the highest score in this simulation result may be set to the reward.

Further, the reward may also be set as a difference between a score at a current time t and a score at a time t−1 of 1-unit time behind, without using the score itself acquired as described above. In the present disclosure, the score is not specified, and the acquired state can be appropriately restored to the molecular graph, so that various indices regarding the molecule and the like can be used as the scores.

As described above, the method of acquiring the reward may be realized based on at least one of the generated latent representation, the tree representation with site information, and the assembled molecular graph. As described above, the reward is cited as a not-limited example, and another reward may also be set according to purposes. For example, when a molecular structure is desired to be inferred as a catalyst, activation energy may be set to the reward. Other than the above, various chemical and physical amounts capable of being used for general molecules and amounts capable of being calculated and acquired from these amounts, can be set to the rewards.

Next, a model used for calculating the policy and the value will be explained. In the present disclosure, a case of using EdgeTreeGRU as a result of improving Tree GRU (Tree Gated Recurrent Unit) will be explained, but the model can be implemented similarly with the use of Tree LSTM (Tree Long Short Term Memory), for example. These are only examples, and the other appropriate network formation method and optimization method may be adopted.

For example, in FIG. 9, the state sₜ is described as the tree representation with site information, this state is input in the policy function to acquire the action by the Actor, and this state is input in the value function to acquire the value by the Critic. The TD error is calculated from the value V(s) obtained based on the value function, and based on this error, the parameters of the policy function and the value function are updated to execute the training. For this reason, the policy function used by the Actor and the value function used by the Critic are desirably formed as neural network models in which the latent representation in which the tree representation with site information is encoded is input.

Here, a hidden vector as the entire tree of the tree representation with site information is set to h(tree), and a latent representation for each node is set to h(node). For example, the h(tree) is an amount generated by concatenating the h(node) regarding each node.

In the present embodiment, as the policy function and the value function, a part thereof is shared in the form of neural network model in which the latent representation can be input, as an example. The training of the shared neural network model can be represented by the following equations.

hᵢⱼ=EdgeTreeGRU(xᵢ,{eᵢⱼ}_{k∈N(i)\j},{hᵢⱼ}_{k∈N(i)\j}) (eq. 1)

kᵢⱼ=Σ_{k∈N(i)\j}[hₖᵢ,eₖᵢ] (eq. 2)

zᵢⱼ=σ(W^{z}xᵢ+U^{z}kᵢⱼ+b^{z}) (eq. 3)

rᵢⱼ=σ(W^{r}xᵢ+U^{r}[hᵢⱼ,eᵢⱼ]+b^{r}) (eq. 4)

mᵢⱼ=tan h(Wxᵢ+UΣ_{k∈n(i)\j}rₖᵢ⊙[hₖᵢ,eₖᵢ]) (eq. 5)

hᵢⱼ=(1−zᵢⱼ)⊙kᵢⱼ+zᵢⱼ└mᵢⱼ (eq. 6)

The neural network that converts the tree representation with site information into the hidden vector is formed by using the EdgeTreeGRU in the above equation (1).

Next, network models to be the functions of the policy and the value, respectively, will be explained.

The EdgeTreeGRU in the equation (1) is GRU designed to input/output the tree representation with site information as a message. x is a vector indicating a feature amount indicating the type or the like of the node, and is expressed by a description based on a one-hot vector, for example. The description based on the one-hot vector indicates a description describing that the one-hot vector is embedded in the variable x used as an argument of the EdgeTreeGRU, for example. e is information on an edge, namely, a vector indicating a feature amount of the site information (including the site direction information), and is expressed by a one-hot vector, for example. h is a message vector, and is described by the tree representation with site information, as described above. By defining a feature amount vector including the site information and giving the message vector between the nodes as a hidden vector of the GRU as above, the processing is executed similarly to the GRU.

Further, σ( ) in each equation indicates a sigmoid function, and odot indicates a product of elements. W, U represent weights, and b represents a bias term.

According to the equation (1) to the equation (6), the message vector h is calculated. Here, the site information is calculated while being concatenated so that the message vector of the GRU includes the information as indicated in the equation (2), the equation (4), and the equation (5).

The condition of stop being one of the actions is desirably judged based on all nodes and edges of the tree representation with site information. Further, the other three actions are desirably judged by referring to the respective nodes. Accordingly, the model used as the policy function no includes a model of acquiring the stop condition from the h(tree) and a model of acquiring the target node, the word, and the site information from the h(node).

The policy function is formed as a neural network model that outputs, when the state in the tree representation with site information is input therein, whether or not the processing is terminated as a binary value, and that outputs the action as a result of selecting the output inferred as the probability distribution via an activation function such as a softmax function (the selection of target node, the selection of word, and the selection of site information), for example.

Further, the model of forming the policy function may be trained through supervised learning. By training the policy through supervised learning, it is possible to improve the possibility of acquiring an appropriate molecular graph according to purposes. For example, in the field of development of new drugs, training is executed by using a data set of a drug-like molecular structure as supervised data. In another field, a data set suitable for purposes of inference in the field is used.

In this training, π_{θ}[aₜ|sₜ] is optimized so as to increase the probability distribution capable of being acquired by the supervised data. By performing the learning as described above, a possibility of inferring a desirable molecule in the reinforcement learning is improved. For this training, a general machine learning method can be used. Further, this training may be executed in advance before the reinforcement learning, or it may be executed in parallel with the reinforcement learning.

The data set is converted into the tree representation with site information, and appropriately input in the neural network model to execute the training.

The value V(s) is desirably judged from all nodes and edges of the tree representation with site information. Accordingly, the model used as the value V(s) further includes a model of acquiring the value from the h(tree). This model may be, for example, MLP (Multi-Layer Perceptron).

As described above, the state sₜ is information regarding the tree representation with site information. As above, the variable input in the policy may be information regarding the tree representation with site information. The information output from the policy may be the probability distribution capable of being obtained from each action. The same applies to the value. The definition of the reward may have a form capable of appropriately calculating the reward with respect to these representations.

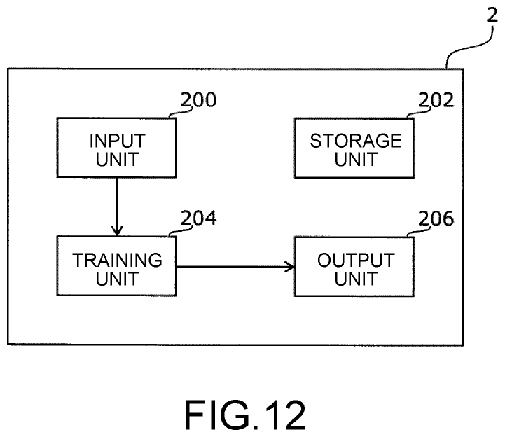

FIG. 12 is a block diagram illustrating one example of a training device that realizes the above-described training. A training device 2 includes an input unit 200, a storage unit 202, a training unit 204, and an output unit 206. The training device 2 is a device that trains the neural network model used for the inferring device 1. Note that the training device 2 may further include another element that is not illustrated, in order to realize an operation.

The input unit 100 includes an input interface, for example, and accepts an input with respect to the training device 2. The input information is, for example, data that is required for training such as supervised data for training the model of the policy. This data may include dictionary data or the like of a molecule that is required for acquiring the action in the policy. Further, this data may include data to be a starting node. The training device 2 executes reinforcement learning in which the action is executed based on the policy from the data to be the starting node to successively update the molecular graph, and based on this update, parameters of the respective neural network models are optimized.

The storage unit 202 stores information that is required for the operation of the training device 2. The storage unit 202 stores at least one of, for example, information such as hyperparameters and parameters required for the training, information obtained in the interim process of the inference, and information such as a program required for the operation of the training device 2. Accordingly, although not illustrated, the storage unit 202 is appropriately connected to each block that transmits/receives data.

The training unit 204 executes, based on the information accepted by the input unit 200, the training of the respective neural network models used for the inference.

The output unit 206 outputs the information such as the parameters optimized by the training unit 204, to the outside or the storage unit 202.

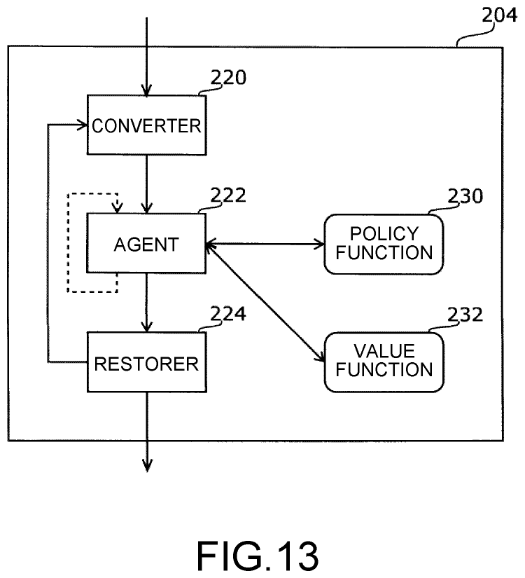

FIG. 13 is a block diagram illustrating one example of the training unit 204. The training unit 204 includes, for example, a converter 220, an agent 222, and a restorer 224.

The converter 220 performs tree decomposition on information or the like regarding a graph of the molecule or the like input in the training unit 204, to thereby acquire the tree representation with site information.

The agent 222 trains a policy function 230 and a value function 232 through reinforcement learning. The agent 222 includes the Actor and the Critic, as illustrated in FIG. 9, for example. The Actor decides an action aₜ based on a policy, and applies the action to an environment, to thereby generate a new state sₜ. To this action, a reward rₜ₊₁ is given. Further, the Critic calculates a value V(s) of the current state based on the state and the reward rₜ₊₁, and calculates a TD error based on this value.

The Actor and the Critic update the policy function 230 and the value function 232 based on this TD error.

After this update, an action is calculated based on a new policy, and processing of training is executed. Note that the update of parameters by the Actor and the Critic may be executed every time the action is decided a predetermined number of times or the state is updated. Timings of these pieces of processing may be realized by a general reinforcement learning method.

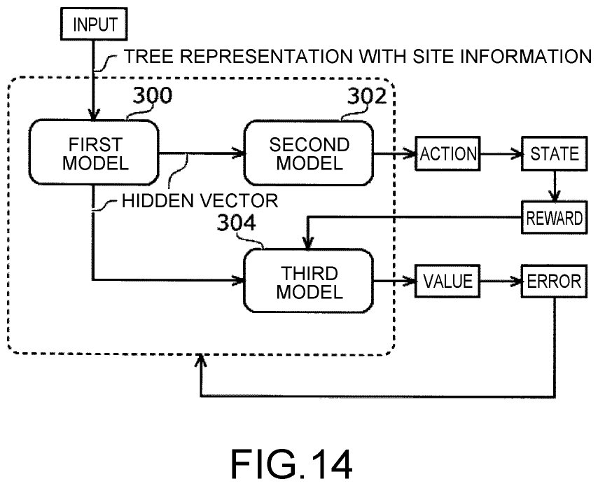

FIG. 14 is a diagram illustrating one example of a configuration of the policy function and the value function according to one embodiment. As illustrated in FIG. 14, the training device 2 executes training of neural network models of a first model 300, a second model 302, and a third model 304, for example.

The first model 300 (encoder) is a model used in a shared manner by the policy function and the value function. This model executes, when information regarding the tree representation with site information is input therein, message passing using a hidden vector corresponding to the tree representation with site information, and converts the information into a description suitable for outputting the action and the value. The first model 300 may be configured as a network that performs conversion from the equation (1) to the equation (6), for example.

The second model 302 is formed as a network that allows an amount output by the first model 300 to be input therein, and outputs each of the above-described actions or the probability distribution of the action. By the action selected based on the action or the probability distribution output by the second model 302, the state is generated from the environment. When the environment is described by the tree information with site information, it is possible to judge whether or not this tree representation with site information is appropriately converted into the graph representation, at an appropriate stage.

Based on the action decided by the agent, the training device 2 generates a new state from the environment, and calculates a reward regarding this state.

The third model 304 allows this reward and the output of the first model 300 to be input therein, and outputs a value. The agent calculates a TD error based on this value, and updates the parameter regarding at least one of the first model 300, the second model 302, and the third model 304.

This update is executed by a plurality of threads, as illustrated in FIG. 9, and for each predetermined episode, the parameters being the copying sources are updated based on the gradients calculated by the respective threads.

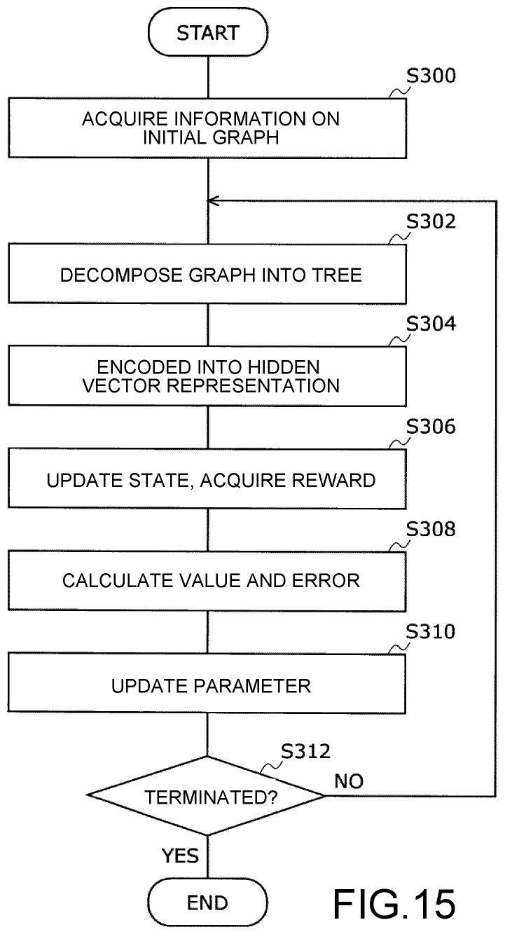

The update of the respective neural network models is executed in a manner as above. FIG. 15 is one example of a flow chart illustrating processing of the training device 2. FIG. 15 explains, as a flow chart, the flow of the processing described in FIG. 9.

First, the training device 2 acquires information on an initial graph via the input unit 200 (S300). Other than the above, when supervised learning is performed in the first model or the like, training data to be supervised data may be input in the training device 2.

Next, the converter 220 performs tree decomposition on the molecular graph, to thereby acquire the tree representation with site information (S302).

Next, the first model 300 converts the tree representation with site information into a hidden vector representation (S304). This conversion is executed based on the equation (1), for example. Further, the message passing is executed based on the equation (2) to the equation (6).

Next, the agent 222 decides the action via the second model 302, and updates the state based on this action (S306). Further, the training device 2 acquires the reward regarding the updated state.

Next, via the third model 304, the agent 222 calculates the value from the reward and the state, and acquires the TD error from this value (S308).

The training device 2 updates the parameters of the respective models based on the TD error (S310). As described above, the update of the parameters may be executed after the calculation of the actions of the predetermined number of times, the state, the reward, or the value. It is also possible to execute supervised learning of the first model 300, the second model 302, and the third model 304, separately from the update of the parameters.

The training device 2 determines whether or not the termination condition has been satisfied (S312), and when the termination condition has not been satisfied (S312: NO), the processing from S302 is repeated. The processing from S302 is executed based on the acquired state, for example. When the termination condition has been satisfied (S312: YES), the parameters are output to the storage unit 202 or the like, and the processing of the training is completed.

The respective models trained as above are used as the trained models of the inferring device 1. Further, as described above, it is possible to execute the reinforcement learning while performing the inference as the inferring device, and in this case, the configuration of the training device 2 can also be used as the inferring device 1.

As described above, according to the present embodiment, it becomes possible to realize the reinforcement learning using the tree representation with site information being a molecule generation method on a graph base. By using the tree representation with site information, it is possible to avoid a deterioration in efficiency due to the generation of invalid molecules. At the same time, it is guaranteed that the molecular structure indicating the state at the middle stage of the episode is a valid molecular structure. Accordingly, it is possible to set a reward using a score capable of being used for general molecules, as the reward, and to set the value. As a result of this, it becomes possible to form a model that enables to easily generate a drug-like molecular structure in the field of development of new drugs, for example. Further, in the field of development of new drugs, it is possible to execute a docking simulation regarding a three-dimensional structure from the molecular graph, and it is possible to realize the inference and the training of the molecular structure that is likely to be bonded to targeted protein.

Not that the configuration of the neural network model as in FIG. 14 is described as an example, and not limited to this configuration. For example, the first model 300 to the third model 304 may be configured as one network model. On the contrary, for example, in the second model 302, the model of inferring the stop condition and the model of inferring the other actions may be configured as separate models.

It is possible to adopt a concept in which all of the above-described trained models include a model that is trained as explained above and then further distilled by a general method, for example.

Further, in the above-described embodiment, the tree decomposition with site information is used for acquiring the tree representation from the graph representation, but not limited to this. Another embodiment can be implemented by appropriate processing capable of converting a graph into tree information, and for processing capable of performing reverse conversion from the tree information into the graph information through the reverse processing, the above-described reinforcement learning method can be used similarly.

Further, although the PPO is described as one method of the reinforcement learning, the method is not limited to this, as described above. For example, in the inferring device 1 and the training device 2, the agents 122, 222 decide the action based on the policy, and the method thereof can be set to any method as described below. As a not-limited example, it is possible to generate a model that calculates, when a state is input therein, a reward and a value for each action capable of being taken with respect to the state. As a not-limited example, it is possible to generate a model that calculates a reward and a value when a state and an action are input therein. As a not-limited example, it is possible to generate a model that outputs a probability distribution of action when a state is input therein, as in the above explanation. As described above, the decision of action in the present disclosure can include both the decision of action itself and the decision of action by selecting it from the probability distribution of action.

As can be understood from this, in the embodiments of the present disclosure, it is possible to use the model appropriately trained through the reinforcement learning by using the tree representation with site information and the model trained through not only the inference but also the reinforcement learning, without depending on the reinforcement learning method. For example, a part or all of the above-described first model 300, second model 302, and third model 304 may be a model trained through the reinforcement learning (reinforcement learning model). Besides, at least a part of the first model 300, the second model 302, and the third model 304 may be a model trained beforehand through supervised learning or unsupervised learning.

When the PPO is used for the training, the model sometimes falls into a local optimum solution as a step of searching proceeds. When the model falls into the local optimum solution, an action output by a policy may be fixed and the model sometimes outputs only the same molecules. When this state is created, entropy of a policy distribution is often converged to 0. For example, at the begging of searching, the model appropriately outputs various pieces of molecular information, but as the step proceeds, it sometimes stops outputting new molecular information.

It is desirable that the model does not fall into such a state and performs searching capable of performing sampling of various kinds of molecules with high reward.

Accordingly, when performing the reinforcement learning by the above-described PPO, processing of regularizing the entropy of the policy may be added. The policy has the maximum entropy when all actions are highly likely to occur at the same probability. On the contrary, when the probability of one action of the policy is dominant, the entropy becomes small. Accordingly, by multiplying the entropy by a coefficient and adding the resultant to a loss, it is possible to realize searching through the reinforcement learning so that the selection probability of one certain action does not become dominant.

The larger the entropy coefficient is, the more various searching can be made, but an excessively large entropy coefficient creates a state closer to random sampling than the above. Further, by appropriately adjusting the entropy coefficient, a reduction speed of entropy can be lowered, but the entropy becomes 0 in the end, and becomes in a fixed state.

In one embodiment, by giving a penalty to a reward, searching with appropriate regularization is realized.

For example, in the reinforcement learning, it is determined whether or not the molecular structure output by the final step of the episode has been already acquired. For example, the inferring device 1 or the training device 2 converts the output molecular structure into a SMILES character string representation, and determines whether or not the SMILES character string representations have been matched.

Further, when predetermined number of the same molecules are output, the inferring device 1 or the training device 2 may set the reward acquired in the episode to 0. The processing of penalty with respect to this reward may be executed in the processing of S306 in FIG. 15, for example.

Further, other than the above, the reward may be set based on the number (number of times) of the acquired same molecule (molecular graph). For example, the penalty may be set in stages with respect to the reward based on the number of the same molecule.

More concretely, it is possible to perform the determination by using, not the equivalence of the generated SMILES, but the equivalence as molecules. The determination using the equivalence as molecules can be executed by using, for example, a canonical SMILES representation of molecule or a fingerprint.

As a not-limited example, it is possible to design such that the reward is set to αⁿ times the original reward (a constant of 0<α<1) at an n-th time, and is attenuated exponentially. In this case, it is also possible that the reward is attenuated in an annealing manner and the reward is increased stochastically according to a predetermined parameter. Further, as another not-limited example, it is possible that the reward is set to 0 and then the reward is restored according to the number of times at which other molecules are searched.

By regularizing the entropy while giving the penalty to the reward as described above, appropriate searching is continued even after the reward is increased. As a result of this, the searching by the model is less likely to fall into the local optimum solution, and thus it is possible to realize searching for a molecule capable of acquiring a better reward.

It is also possible to generate a reinforcement learning model by using the training device 2 or the above-described training method in the present disclosure.

Further, it is also possible to generate a molecular structure by the inferring device 1 using the generated reinforcement learning model or the above-described inferring method using the reinforcement learning model.

The trained models of above embodiments may be, for example, a concept that includes a model that has been trained as described and then distilled by a general method.

Some or all of each device (the inference device 1 or the training device 2) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

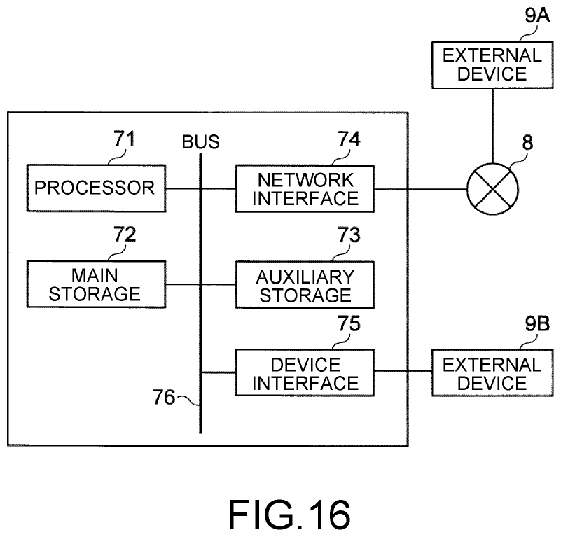

FIG. 16 is a block diagram illustrating an example of a hardware configuration of each device (the inference device 1 or the training device 2) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 16 is provided with each component one by one but may be provided with a plurality of the same components. Although one computer 7 is illustrated in FIG. 16, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the inference device 1 or the training device 2) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the inference device 1 or the training device 2) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the inference device 1 or the training device 2) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the inference device 1 or the training device 2) in the above embodiments may be enabled by the main storage device 72 or the auxiliary storage device 73 or may be implemented by a built-in memory built into the processor 71. For example, the storages 102, 202 in the above embodiments may be implemented in the main storage device 72 or the auxiliary storage device 73.

In the case of each device (the inference device 1 or the training device 2) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the inference device 1 or the training device 2) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An inferring device comprising:
one or more memories; and
one or more processors configured to:
decide an action based on a tree representation including a node and an edge of a molecular graph, and a trained model trained through reinforcement learning; and
generate a state including information on the molecular graph based on the action, wherein
the edge has connection information on the nodes.

2. The inferring device according to claim 1, wherein
the one or more processors are configured to:
convert the tree representation into a hidden vector; and
input the hidden vector in the trained model to decide the action.

3. The inferring device according to claim 1 wherein
the one or more processors are configured to decide, as the action, at least any one of
a first action indicating to which node out of the nodes a new node is connected,
a second action indicating information on the new node,
a third action indicating connection information on the new node, or
a fourth action indicating whether or not inference is continued.

4. The inferring device according to claim 1, wherein
the one or more processors are configured to execute reinforcement learning of the trained model.

5. The inferring device according to claim 4, wherein
the one or more processors are configured to:
calculate a reward regarding the state generated by the action; and
update the trained model based on the reward.

6. The inferring device according to claim 5, wherein
the one or more processors are configured to calculate the reward based on information regarding a molecule corresponding to the state.

7. The inferring device according to claim 6, wherein
the one or more processors are configured to decide the reward based on the number of times of acquisition of the same molecular graph.

8. The inferring device according to claim 7, wherein
the one or more processors are configured to give a penalty to the reward when a predetermined number of the same molecular graphs are acquired.

9. The inferring device according to claim 8, wherein
the one or more processors are configured to set the reward to 0 when the predetermined number of the same molecular graphs are acquired.

10. A training device comprising:
one or more memories; and
one or more processors configured to:
decide an action by inputting information regarding a tree representation including a node and an edge of a molecular graph in a reinforcement learning model;
generate a state including information on the molecular graph based on the action; and
update the reinforcement learning model based on the state, wherein
the edge has connection information on the nodes.

11. The training device according to claim 10, wherein
the reinforcement learning model is a model trained in advance through supervised learning.

12. The training device according to claim 10, wherein
the one or more processors are configured to:
calculate a reward regarding the state; and
update the reinforcement learning model based on the reward.

13. The training device according to claim 12, wherein
the one or more processors are configured to calculate the reward based on information regarding a molecule corresponding to the state.

14. The training device according to claim 13, wherein
the one or more processors are configured to decide the reward based on the number of times of acquisition of the same molecular graph.

15. The training device according to claim 14, wherein
the one or more processors are configured to give a penalty to the reward when a predetermined number of the same molecular graphs are acquired.

16. The training device according to claim 15, wherein
the one or more processors are configured to set the reward to 0 when the predetermined number of the same molecular graphs are acquired.

17. The training device according to claim 13, wherein
the reward is a fat-soluble score.

18. The training device according to claim 13, wherein
the reward is a docking score.

19. The training device according to claim 10, wherein
the one or more processors are configured to:
convert the tree representation into a hidden vector; and
input the hidden vector in the reinforcement learning model to decide the action.

20. An inferring method comprising:
deciding, by one or more processors, an action based on a tree representation including a node and an edge of a molecular graph, and a trained model trained through reinforcement learning; and
generating, by the one or more processors, a state including information on the molecular graph based on the action, wherein
the edge has connection information on the nodes.
